(19) United States
(12) Patent Application Publication
(10) Pub. No.: US 2017/0072007 A1
(43) Pub. Date: Mar. 16, 2017

(54) PHANTOM PHENOMENA TREATMENT

(71) Applicant: OTOLANUM AG, Zug (CH)

(72) Inventors: Marlies KNIPPER-BREER, Stetten (DE); Lukas Rüttiger, Kusterdingen (DE)

(73) Assignee: Otolanum AG

(21) Appl. No.: 15/343,817

(22) Filed: Nov. 4, 2016

Related U.S. Application Data
(63) Continuation of application No. 14/572,376, filed on Dec. 16, 2014, now abandoned.
Application No. 14/572,376 is a continuation of application No. 13/526,185, filed on Jun. 18, 2012, now abandoned.
Application No. 13/526,185 is a continuation-in-part of application No. 11/814,663, filed on Feb. 13, 2008, now abandoned.

(30) Foreign Application Priority Data
Jan. 25, 2005 (DE) DE102005004343.7

(51) Int. Cl. A61K 38/12; C12Q 1/68; A61K 9/00

(52) U.S. Cl. CPC A61K 38/12; A61K 9/0046; C12Q 1/6876; C12Q 2600/158

(57) ABSTRACT

The present invention relates to a substance for the treatment of the phantom phenomena of tinnitus and/or phantom pain, a method for the diagnosis and for the treatment of these phantom phenomena.

### CROSS REFERENCE TO RELATED APPLICATIONS

This application is a continuation-in-part of U.S. patent application Ser. No. 11/814,663, which is a U.S. National stage of International Patent Application PCT/EP2006/000446 filed Jan. 19, 2006, and claims priority of German Patent Application DE 10 2005 004 343.7 filed Jan. 25, 2005, which is incorporated herein by reference.

### FIELD OF THE INVENTION

The present invention relates to a substance for treating phantom phenomena, specifically tinnitus, such as acute, subacute, and/or chronic tinnitus, and/or phantom pain, a method for the diagnosis and for the treatment of these phantom phenomena.

Substances and methods of these types are generally known in the state of the art.

The phantom phenomenon of tinnitus means the noises which are perceived by a patient and which are generated by the ear and the auditory system. Tinnitus which has existed for only a few weeks and up to three months is referred to as acute tinnitus. If the tinnitus exists for more than three months up to 12 months, it is referred to as subacute, and if the tinnitus exists for more than one year, it is referred to as chronic. Epidemiological enquiries show that about three million adults, i.e. about 4% of the population, in Germany are affected by chronic tinnitus. Considered globally, each year about ten million people experience tinnitus, which develops from an acute into a chronic form in about 340 000 cases, called the new case rate.

The diverse causes of tinnitus include chronic noise damage, acute loud-noise injury to hearing, sudden loss of hearing and other types of disorders associated with loss of hearing. Connections with inner ear hearing impairment as chronic-progressive form or as noise-induced hearing impairment followed by Menière's disease and loss of hearing are, according to clinical studies, connected with tinnitus in more than two out of three. Disorders of the cervical spine and of the mandibular joint and masticatory muscle system are also involved in the development and persistence of tinnitus. Tinnitus appears also to have a psychological component, so that reference is made to psychogenic tinnitus in this connection. However, despite intensive diagnostic investigation, no certain cause of tinnitus is evident in many cases.

At present, tinnitus therapy makes use of psychosomatic treatment, relaxation therapy, biofeedback, hypnotherapy, electrical stimulation, lidocain, iontophoresis or masking. However, these are exclusively symptomatic therapeutic policies.

WO 02/15907 A1 proposes to treat tinnitus by administering the potassium channel opener flupirtine. This treatment has the disadvantage that flupirtine is additionally a muscle-relaxing analgesic, and thus administration would be associated with side effects which are not to be tolerated.

Wang et al. (2000), Evaluating effects of some medicine on tinnitus with animal behavioral model in rats, Zhonghua Er. Bi. Yan. Hou. Ke. Za. Zhi. 35 (5), abstract, propose the administration of nimodipine for treating tinnitus. Nimodipine is an inhibitor of the Cav1.3 Ca⁺⁺ channel. However, it has emerged in this connection that blockade of the Cav1.3 channel in the auditory system would lead directly to deafness, so that nimodipine is entirely unsuitable for treating tinnitus.

WO 2004/022069 A1 describes aberrant NMDA (N-methyl-D-aspartate) receptors as one of the possible causes of tinnitus. These altered so-called glutamate receptor channels, which are expressed inter alia by auditory nerve cells, lead to an increased influx of calcium to the cell. It is proposed in this document that NMDA receptor antagonists be used to treat tinnitus. However, it remains entirely unclear whether the acute or chronic tinnitus situation can be treated with these substances. In addition, there are no indications at all about how the substances are to be administered.

DE 101 24 953 A1 proposes a tinnitus treatment policy based on stimulation of the expression of the brain-derived nerve growth factor (BDNF). The authors there describe, on the basis of an animal model, that a reduction in BDNF expression prevails in the cochlea and in the inferior colliculus in chronic tinnitus, for which reasons stimulation of BDNF expression is proposed as therapeutic approach. The authors there have quite specifically and exclusively investigated the situation in chronic tinnitus. Thus, the rats used in the animal model were treated with salicylates for three months, thus inducing chronic tinnitus in a known manner; cf. Penner M. J., and Jastreboff P. J. (1996), Tinnitus: Psycho-physical observations in humans and animal models, in: Van de Water, Popper A. N., Fax, R. R. (Ed.), Clinical aspects of hearing, Springer, New York, Heidelberg, pages 208 to 304; and Bauer, C. A., et al. (1999), A behavioral model of chronic tinnitus in rats. Otolaryngol. Head Neck Surg. 121, pages 457 to 462.

However, the authors of DE 101 24 953 A1 have not realized that there must be significant differences between the treatment of chronic and acute tinnitus.

An overview of the pathological state of tinnitus is to be found for example in Waddell, A., Canter, R. (2004), Tinnitus, Am. Fam. Physician 69, pages 591 to 592.

The phantom phenomenon of phantom pain means the projection of sensations into a part of the body which has been amputated or denervated for example by plexus damage or spinal cord injury, an extremity, the breast, the rectum, a tooth inter alia. This part of the body is perceived to be present and after extremity amputation for example the sensation is of a swollen hand or foot located directly on the stump.

Numerical data on the numbers of cases where phantom pain occurs after amputation are the subject of disagreement and range from 5 to 100%.

Phantom pain is currently treated in the framework of pain therapies for example with anticonvulsants, baclofen or calcitonin. Occasionally, assistance is provided by pain-relieving antidepressants. Surgical methods are also used for example to block or stimulate nerves. However, no targeted, causal treatment method yet exists, especially because the underlying molecular mechanisms are not completely understood.

An overview of the pathological state of phantom pain is to be found in Middleton, C. (2003), The causes and treatments of phantom limb pain, Nurs. Times 99, pages 30 to 33.

It is therefore an object of the present invention to provide a substance or a therapeutic policy with which the disadvantages of the prior art are prevented. It is intended in particular to provide such a substance enabling targeted treatment of the phantom phenomena of tinnitus, such as acute, subacute, and/or chronic tinnitus, and of phantom pain.

This object is achieved by the provision of the substance which interacts with the BDNF signal transduction cascade.

BDNF (brain-derived nerve growth factor) is a basic protein which is produced by neurons of the central nervous system and consists of 252 amino acids. BDNF is a growth factor involved in the development of the nervous system and playing a part inter alia in the development of the plasticity of synapses. The effect of BDNF is mediated via specific receptors, for example via the BDNF receptor trkB which in turn regulates downstream factors such as MAP kinase or Cam kinase in terms of their activity or mode of action. BDNF in turn is itself regulated, for example by calcium.

All the factors which influence or regulate the activity, expression, mode of action or the like of BDNF, and all the factors which are influenced or regulated in this way by BDNF form the so-called BDNF signal transduction cascade.

The BDNF signal transduction cascade can be divided into a cascade upstream of the BDNF receptor trkB, and one downstream thereof. The signal transduction cascade downstream of trkB is initiated by the binding of BDNF and other members of the neutrotropin family to the trk receptor. This leads to trk dimerization and activation of the tyrosine kinase activity of the receptor. This ligand-mediated aggregation of the receptors results in autophosphorylation of intracellular domains which are followed by activation of signal molecules such as phospholipase C (PLC), phosphatidylinositol 3-kinase (PI3 kinase) and the adaptor protein Shc (SH-2-containing protein). The further signals, in particular mediated by the ras-dependent MAP kinase, ultimately influences cellular gene transcription and protein synthesis.

The cascade upstream of trkB relates to the regulation of BDNF. Thus, it is known that BDNF expression is regulated activity-dependently by various stimuli such as electrical stimulation or injury, pure physical movement or else by the circadian rhythm. The aforementioned stimuli regulate the expression of various untranslated BDNF exons which then finally form, together with the common 5′ exon, various BDNF transcripts. The stimuli apparently act via different Ca⁺⁺-induced signal cascades on the promoters of the various BDNF exons.

The BDNF signal transduction cascade means according to the invention both the cascade downstream of trkB and that upstream of trkB. It will be appreciated that trkB itself is also a component of the BDNF signal transduction cascade.

An overview of the BDNF signal transduction cascade is to be found in Gabellini, N. (2004), Transcriptional regulation by cAMP and Ca⁺⁺ links the Na⁺/Ca⁺⁺ exchanger 3 to memory and sensory pathways, Mol. Neurobiol. 30, pages 91 to 116; West et al. (2001), Calcium Regulation of Neuronal Gene Expression, Proc. Natl. Acad. Sci. USA 98, pages 11024-11031. The contents of these publications are incorporated into the present application by reference.

BDNF is known to play a part in a large number of diseases; cf. Binder, D. K. (2004), The role of BDNF in epilepsy and other diseases of the mature nervous system, Recent Advances in Epilepsy Research, pages 34 to 56.

A substance which interacts with the BDNF signal transduction cascade can be represented in any form, i.e. it may be such a substance which is defined chemically, biochemically or biologically, and thus which is any embodiment of a chemically synthesized compound, which represents a molecule, ion, atom, a protein, peptide, antibody, a nucleic acid, an aptamer, a virus, bacterium, etc.

Interaction means according to the invention the direct or indirect interaction of this substance with a factor of the BDNF signal transduction cascade, which results in a modification of the physiological signal transduction within this cascade. Such interacting substances are adequately described in the state of the art.

The object of the present invention is completely achieved by the provision of such a substance. In particular, the inventors have provided for the first time a common therapeutic policy for the phantom phenomena of tinnitus, such as acute, subacute, and/or chronic tinnitus, and of phantom pain.

Thanks to the invention, a therapy of tinnitus, particularly of acute tinnitus, in a targeted manner is provided and thus it is possible to prevent the problem of this phantom phenomenon becoming chronic. In the state of the art there is frequently no differentiation made between chronic and acute tinnitus, and in many cases merely approaches to the treatment of chronic tinnitus are proposed, as also in the abovementioned DE 101 24 953 A1.

The present invention provides a therapy for all stages of tinnitus, such as for acute, subacute, and/or chronic tinnitus. In particular, the present invention provides a method for treating acute, subacute and/or chronic tinnitus, as well as a method for preventing subacute, and/or chronic tinnitus.

As the inventors have further found, the concept of interaction with the BDNF signal transduction cascade can also be applied to the treatment of phantom pain.

It is preferred in this connection according to the invention to provide an interacting substance which brings about blockade or inhibition of the BDNF signal transduction cascade.

In this sense according to the invention, blockade or inhibition means that the signal transmission within the BDNF signal transduction cascade is retarded, worsened, reduced or completely suppressed compared with the physiological situation.

This realization by the inventors was completely surprising and contrasts with the findings described in DE 101 24 953 A1 for chronic tinnitus. Thus, the aforementioned document specifically suggests stimulation of BDNF expression, whereas the invention suggests treating the phantom phenomena tinnitus, particularly acute tinnitus, by inhibiting BDNF expression or BDNF signal transduction. The inventors have therefore realized that the therapy necessary for acute tinnitus may deviate in form to that proposed for chronic tinnitus in the aforementioned document. Application of this known concept to the acute state would therefore have fatal consequences.

The inventors of the present invention have induced acute tinnitus in rats by a short-term administration of salicylates and found an enhanced expression of BDNF in the cochlear ganglia.

It has thus surprisingly emerged that differentiation between acute and chronic tinnitus is essential for choosing the correct treatment method, and has to date not taken place, or taken place inadequately, in the state of the art. Tinnitus, in particular acute tinnitus, is therefore to be treated according to the invention by inhibition or blockade of the BDNF signal transduction cascade, for example by inhibiting BDNF expression.

An analogous treatment is proposed by the inventors for phantom pain.

The inventors present here for the first time molecular data indicating the mechanisms of the pathology of tinnitus and phantom pain, and thus provide the basic sciences and medicine with a better understanding of these phantom phenomena and, at the same time, indicate a causal therapeutic policy.

A substance which is preferably provided according to the invention is one which brings about blockade or inhibition of the signal transduction cascade upstream of trkB. Such substances are suitable for treating tinnitus at any stage, such as acute tinnitus, subacute tinnitus, and chronic tinnitus, and phantom pain.

According to this advantageous approach, BDNF activation or expression either does not take place at all or else is reduced, and BDNF cannot transmit its signals to trkB. Or else further factors located upstream from trkB are influenced in their activity in such a way that they cannot transmit their signals to trkB.

Substances which inhibit factors of the BDNF signal transduction cascade can easily be found by activity or inhibition assays which form part of the routine activities of a molecular biologist or of a clinical chemist or pharmacologist. Inhibiting substances upstream of trkB which are preferred according to the invention relate to L-type Ca⁺⁺ channel antagonists, advantageously selected from the group consisting of nicardipine, nifedipine, and isradipine, and CREP antagonists and glutamate antagonists.

As the inventors have been able to find, such substances are particularly suitable as pharmacological active ingredients in a medicament for the treatment of tinnitus, such as acute, subacute, and/or chronic tinnitus, or phantom pain, or for the prevention of chronic tinnitus. The signal protein CREP (cAMP response element binding protein) leads via interaction with Ca⁺⁺ and cAMP together with glutamate to stimulation of BDNF expression via activation of distinct BDNF promoter regions. Interruption of this cascade by glutamate receptor antagonists or substances which prevent for example cAMP kinase or Ca⁺⁺-calmodulin-dependent kinase-mediated (CaMK) phosphorylation of CREB, thus inhibit BDNF expression, and acute tinnitus or phantom pain can be effectively treated. CREP antagonists suitable for this purpose are for example H89 as cAMP kinase inhibitor, and KN-93 as CaNK inhibitor.

It is preferred according to the invention to use a GABA receptor agonist, preferably a benzodiazepine or a substance related thereto, as substance interacting with the BDNF signal transduction cascade upstream of trkB. These preferably include midazolam, diazepam, flurazepam, oxazepam, nitrazepam, flunitrazepam, clonazepam, triazolam, clobazam and brotizolam. GABA receptor agonists further preferably used are baclofen, gamma-vinyl-GABA, gamma-acetylene-GABA, progabide, muscimol, iboten, sodium valproate or tetrahydroisoxazolopyridine (THIP).

The inventors have been able to show that the aforementioned substances are particularly suitable for the treatment of tinnitus, such as acute, subacute and/or chronic tinnitus, and/or of phantom pain. Thus, rats in which acute tinnitus and an enhanced expression of BDNF in the cochlear ganglia was induced by administration of salicylates were given various GABA receptor agonists such as, for example, the benzodiazepine midazolam. It emerged from this that BDNF expression in the cochlear neurons, and thus the symptoms of acute tinnitus, were significantly reduced by this administration. The aforementioned substances therefore act as BDNF antagonists and interact, surprisingly, with the BDNF signal transduction cascade.

The invention provides alternatively a substance which brings about a blockade or inhibition of the BDNF receptor (trkB) or of the signal transduction cascade downstream thereof.

This measure has the advantage that a further suitable point of attack is utilized for a therapeutic intervention. Thus, trkB itself can be blocked by means of suitable substances known in the state of the art, and thus the entire subsequent BDNF signal transduction cascade. A further possibility is to develop by means of molecular drug design on the basis of crystallographic data for trkB suitable substances interacting with this receptor which represent advantageous active ingredients for a medicament for the treatment of tinnitus, such as acute, subacute and/or chronic tinnitus, or phantom pain, or for prevention of subacute and/or chronic tinnitus.

Factors downstream of trkB in the signal transduction cascade, such as, for example, PLC, PI3 kinase or Shc, are also suitable points of attack for a use according to the invention of the substance. Shutting down or inhibition of these factors likewise leads to a suitable blockade of the BDNF signal transduction cascade.

It is preferred in this connection to provide as substance a MAP kinase inhibitor, a Cam kinase inhibitor or a trkB antagonist. Particularly suitable MAP kinase inhibitors according to the invention are the substances U 0126 or PD 98058.

In a particularly preferred embodiment, the substance is a trkB antagonist which preferably blocks or inhibits the BDNF receptor (trkB). For example, the substance may block trkB by directly binding to trkB, for example, by binding to a functional site on trkB, such as to the BDNF binding site. The trkB antagonist may also inhibit the natural trkB ligand (e.g., BDNF or other members of the neutrotropin family) from binding to the receptor, for example, by binding to the BDNF binding site on trkB or by binding to the ligand. Thus, in a particularly preferred embodiment, the substance is a trkB antagonist, preferably inhibiting the natural ligand from binding to the receptor, for example, by binding directly to trkB or to the ligand. Such a trkB antagonist may be any suitable substance, such as a small molecule, a peptide, a protein, an antibody or a fragment of an antibody, a nucleic acid, an aptamer etc. In a preferred embodiment, the trkB antagonist does not act upstream or downstream of trkB. This means that the trkB antagonist preferably does not interact directly with any components of the BDNF signaling cascade other than trkB or trkB ligands (such as BDNF or other members of the neutrotropin family).

As the inventors have found out, such substances are particularly suitable for the treatment of tinnitus, such as acute, subacute, and/or chronic tinnitus, and phantom pain. U 0126, MEK1/2 inhibitor and PD 98058, an MEK1 inhibitor, can be purchased from Cell Signalling Technology, Inc., Beverly, Mass., United States of America. In this connection, the choice of the concentration employed is up to a person skilled in the art and depends on the severity of the disease, the remaining therapeutic policy, and various individual factors from the patient to be treated. With this background, the concentration employed will be established for the particular individual case by a person skilled in the art using routine measures.

In an advantageous embodiment of the use according to the invention, the substance is administered locally on or in the ear or on the amputation site. For example, the substance may be administered locally on or in the ear using a micrometering system.

This measure has the advantage that the substance is administered in a targeted manner at the site of action, so that only small amounts of active substance are necessary. The stress on the remainder of the treated patient's body is thus less, and side effects are substantially reduced. In the case of treatment of acute tinnitus, the micrometering system described by Lehner, R. et al. (1996), A new implantable drug delivery system for local therapy of the middle and inner ear, Ear. Nose Throat 76, pages 567 to 570, is appropriate.

Local administration of the substance can also take place alternatively by use of biodegradable hydrogel which serves as carrier matrix for the substance. Such biodegradable hydrogel has already been used successfully in the animal model for the local administration of BDNF onto the round window of the inner ear; Ito et al. (2005), A new method for drug application to the inner ear, J. Otorhinolaryngol. Relat. Spec., pages 272-275.

The inventors have found that local administration of the substance interacting with the BDNF signal transduction cascade is particularly advantageous because, as the inventors have been able to find in further experiments, BDNF expression in acute tinnitus is surprisingly reduced in the auditory cortex in contrast to the situation in the cochlear ganglia. Systemic administration of substances inhibiting the BDNF signal transduction cascade would therefore lead to an even further reduction in BDNF expression in the cortex and thus have harmful effects on the organism. Systemic administration of the substance which inhibits the BDNF signal transduction cascade would therefore be contraindicative for acute tinnitus and phantom pain.

Another object of the present invention is a substance for the therapeutic and/or prophylactic treatment of the phantom phenomena of tinnitus, such as acute, subacute, and/or chronic tinnitus, and/or phantom pain in a living being which is selected from the group consisting of: MAP kinase inhibitor, in particular U 0126 or PD 98058, Cam kinase inhibitor, L-type Ca⁺⁺ channel antagonist, in particular nicardipine or nifedipine or isradipine, CREP antagonist, in particular H89 and KN-93, or glutamate antagonist and trkB antagonist.

The inventors have for the first time recognized and proposed a specific medical use of the aforementioned substances in connection with phantom phenomena such as tinnitus, e.g., acute, subacute and chronic tinnitus, and phantom pain.

Another object of the present invention relates to a method for diagnosing the phantom phenomena of acute tinnitus and/or phantom pain in a living being, which comprises the following steps: (a) provision of a biological sample of the creature, (b) determination of the level of expression of BDNF in the biological sample, (c) comparison of the level from step (b) with a reference value from a healthy creature, and (d) correlation of a level lying above that of a healthy creature with a positive diagnosis.

It is possible according to the invention to use any such type of biological sample, but it preferably derives from the ear or from the amputation site, such as a tissue sample, cells, for example cochlear ganglia or nerve cells. However, the biological sample may also be a systemic blood sample; Lang et al. (2005), Association of BNDF serum concentrations with central serotonergic activity; Evidence from auditory signal processing, Neuropsychopharmacology 30 (6), pages 1148-1153. Care must be taken in this connection that when a tissue sample is taken from the ear there is no damage to hearing, it being unnecessary to observe such a precautionary measure if the tinnitus develops centrally to a no longer intact ear. Taking such a sample for determining the level of expression of BDNF can then also be used simultaneously for measuring the functioning of the transmission and utilization of more centrally located nerves in order, for example, to optimize the efficiency of implantation of a cochlear implant.

The method is carried out in a suitable biological system, it being possible to use conventional buffers such as Tris or HEPES buffer. The level of expression is determined in step (b) by conventional methods of molecular and cell biology, such as ELISA techniques and Western blotting at the protein level, or Northern blotting at the mRNA level. Suitable methods of molecular biology are described for example in Sambrook, J. and Russel, D. W. (2001), Molecular Cloning—A Laboratory Manual, 3rd Edition, Cold Spring Harbor Laboratory Press, Cold Spring Harbor, N.Y., the content of this publication is incorporated into the present description by reference.

The inventors have thus succeeded for the first time in establishing and providing a molecular biological diagnosis of acute tinnitus and phantom pain.

Another object of the present invention is a method for the treatment of the phantom phenomena of tinnitus, such as acute, subacute and/or chronic tinnitus, and/or phantom pain in a living being, such as a living animal or human being, which comprises the following steps: (a) provision of a medicament which comprises a substance interacting with the BDNF signal transduction cascade, and a pharmaceutically acceptable carrier and, where appropriate, further excipients and/or active ingredients, and (b) administration, where appropriate locally, of the medicament to the creature, and, where appropriate, (c) repetition of steps (a) and (b).

Pharmaceutically acceptable carriers and further excipients are sufficiently well known in the state of the art, cf. for example Kibbe, A. H. (2000), Handbook of Pharmaceutical Excipients, 3rd Edition, American Pharmaceutical Association and Pharmaceutical Press, the content of this publication is incorporated into the present description by reference.

Further active ingredients which are suitable are for example conventional analgesics or tinnitus remedies.

In summary, the present invention relates to a method for the treatment of the phantom phenomena of tinnitus, such as acute, subacute, and/or chronic tinnitus, and/or of phantom pain in a human or animal being, comprising administering to the human or animal being an effective amount of a substance, which interacts with the BDNF signal transduction cascade thereby treating the phantom phenomena of tinnitus, such as acute, subacute, and/or chronic tinnitus, or of phantom pain in the human or animal being, for example, the substance brings about a blockade or inhibition of the BDNF signal transduction cascade.

In one embodiment, the substance brings about a blockade or inhibition of the signal transduction cascade upstream of the BDNF receptor (trkB), for example, the substance may be (i) a GABA receptor agonist, such as a benzodiazepine or substances related thereto preferably selected from the group consisting of: midazolam, diazepam, flurazepam, oxazepam, nitrazepam, flunitrazepam, clonazepam, triazolam, clobazam and brotizolam, (ii) an L-type Ca⁺⁺ channel antagonist, for example selected from the group consisting of: nicardipine, nifedipine and isradipine, or (iii) CREP antagonists or glutamate antagonists, for example, selected from the group consisting of H89 and KN-93.

In a preferred embodiment, the GABA receptor agonist is a substance selected from the group consisting of: baclofen, gamma-vinyl-GABA, gamma-acetylene-GABA, progabide, muscimol, iboten, sodium valproate and tetrahydroisoxazolopyridine (THIP).

In one embodiment, the substance brings about a blockade or inhibition of the BDNF receptor (trkB) or of the signal transduction cascade downstream thereof, for example, the substance may be (i) a MAP kinase inhibitor, such as U 0126 or PD 98058, (ii) a Cam kinase inhibitor, or (iii) a trkB antagonist.

In a preferred embodiment, the substance is administered locally on or in the ear or at the amputation site.

Furthermore, the present invention relates to a method for diagnosing a phantom phenomena of tinnitus, such as acute, subacute, and/or chronic tinnitus and/or phantom pain in an animal or human being, which comprises the steps (a) providing a biological sample, (b) determining the level of expression of BDNF in the biological sample, (c) comparing the level from step (b) with a reference value from a healthy being, and (d) correlating a level lying above that of a healthy being with a positive diagnosis.

Furthermore, the present invention relates to a method for treating a phantom phenomena of tinnitus, such as acute, subacute, and/or chronic tinnitus, and/or phantom pain in a human being, which comprises the steps (a) providing a medicament which comprises an effective amount of a substance interacting with the BDNF signal transduction cascade, and a pharmaceutically acceptable carrier, and (b) administering the medicament to the human being, wherein preferably administering the medicament occurs locally on or in the ear or at an amputation site of the being. The method may further comprise repeating steps (a) and (b).

In a particularly preferred embodiment, the present invention relates to a method for the treatment of the phantom phenomena of acute, subacute and/or chronic tinnitus in a human or animal being, preferably to the treatment of subacute and/or chronic tinnitus, comprising administering to the human or animal being an effective amount of a substance, which brings about a blockade or inhibition of the BDNF receptor (trkB), preferably an effective amount of a trkB antagonist, thereby treating the phantom phenomena of tinnitus, preferably thereby treating the phantom phenomea of subacute and/or chronic tinnitus, in the human or animal being.

It will be appreciated that the features mentioned above and yet to be explained below can be used not only in the combination indicated in each case, but also in other combinations or alone, without departing from the scope of the present invention.

The invention is now explained in more detail by means of exemplary embodiments which are purely illustrative and from which further features and advantages of the invention are evident.

### BRIEF DESCRIPTION OF THE DRAWINGS

The appended figures depict the following:

FIG. 1: The operation and local supply of salicylate does not significantly alter the hearing thresholds;

FIG. 2 Acute systemic and local administration of salicylate leads after a certain time to an increased c-fos expression in the cochlea;

FIG. 3 In situ hybridization analysis of c-fos and BDNF exon III and exon IV splice variants in the cochlea of adult rats after local and systemic administration of salicylate;

FIG. 4 An RT-PCR analysis shows a dose-dependent differential alteration in expression of c-fos and BDNF exon III and exon IV expression in the cochlea after systemic and local administration of salicylate;

FIG. 5 Local administration of salicylate reduces the expression of c-fos, BDNF exon III and exon IV in the auditory cortex;

FIG. 6 The upregulation caused by salicylate in BDNF expression in cochlear neurons is inhibited by the type Ca⁺⁺ channel antagonist nifedipine; and

FIG. 7 The upregulation caused by salicylate in BDNF expression in cochlear neurons is inhibited by the benzodiazepine midazolam;

FIG. 8 Tinnitus provoked by acoustic trauma (120 dB at 10 kHz for 2 hours) to rats is attenuated by administration of a trkB antagonist.

### DETAILED DESCRIPTION OF EMBODIMENTS

### Material and Methods

### Animals

Adult female Wistar rats weighing between 280 and 300 grams were used for the experiments. The treatment and handling of these animals took place in conformity with the institutional guidelines of Tübingen University, Tierforschungsanstalt.

The rats were anesthetized intraperitoneally by the method of Guitton, M. J. et al. (2003), Salicylate induces tinnitus through activation of cochlear NMDA receptors, J. Neurosci. 23, pages 3944 to 3952, in order to place a so-called gel foam (Gelita Tampon; B. Braun Medical, Melsungen, Germany) over the round window of both ears. The gel foam was impregnated as indicated with salicylate, which was diluted in artificial perilymph solution (70 mg/ml), or with the corresponding volume of artificial perilymph alone. The gel foam was in the niche of the round window as described; Guitton J. et al. (loc. cit.). The salicylate was locally administered thus for 20 hours. After this time, the animals were sacrificed and the cochlea and the auditory cortex were removed.

### Tissue Preparation

Carbon dioxide was used for deep anesthesia of the animals, which were then decapitated. The cochleae were isolated and prepared as described previously; cf. Knipper, M. et al. (2000), Thyroid hormone deficiency before the onset of hearing causes irreversible damage to peripheral and central auditory systems, J. Neurophysiol. 83, pages 3101 to 3112. Briefly, the cochleae were fixed by immersion in 2% paraformaldehyde, 125 mM sucrose in 100 mM phosphate-buffered saline (PBS), pH 7.4, for two hours and, following the fixation, decalcified in rapid bond decalcifier (#904687, Eurobio, Fisher-Scientific, 61130 Nidderau, Germany) for 45 minutes, subsequently followed by incubation in 25% sucrose, 1 mM protease inhibitor (Pefabloc, Roche) in Hanks buffered saline overnight. Following the overnight incubation, the cochleae were embedded in O.C.T. compound (Miles Laboratories, Elkhart, Ind., USA). The tissues were then cryosectioned with a thickness of 10 μm for the in situ hybridization, stored on SuperFrost*/plus slides and stored at −20° C. before use.

The auditory cortices were identified according to the descriptions given in Paxinos, G. & Watson, C. (1998), The rat brain in stereotaxic coordinates, Academic Press, Inc. For RNA preparations, the tissues were frozen directly in liquid nitrogen and stored at −70° C. before use.

### Riboprobe Synthesis and In Situ Hybridization

Genomic DNA from rat liver tissue was isolated using the Easy DNA kit from Invitrogen in accordance with the manufacturer's information. A polymerase chain reaction was carried out in order to amplify all four non-coding 5′ exons of the BDNF gene.

The exon-3-specific probe was amplified by using a sense primer (5′ acc cac ttt ccc att cac cg 3′) and an antisense primer (5′ cct ttt tca gtc act act tg 3′) in each case corresponding to nucleotide positions 536 to 555 and 957 to 976 of genomic fragment B (Timmusk, T. et al. (1995), Identification of brain-derived neurotrophic factor promoter regions mediating tissue-specific, axotomy-, and neuronal activity-induced expression in transgenic mice, J. Cell. Biol. 128, pages 185 to 199). For the exon-4-specific probe, a sense primer (5′ cca atc gaa gct caa ccg aa 3′) and an antisense primer (5′ tca ggg tcc aca caa agc tc 3′) corresponding to nucleotide positions 1732 to 1751 and 2059 to 2078 of genomic fragment B was used (Timmusk, T. et al., loc. cit.). In order to amplify the common riboprobe for the coding exon-5, a sense primer (5′ gag gac cag aag gtt cg 3′) and an antisense primer (5′ ttt atc tgc cgc tgt gac 3′) corresponding to nucleotide positions 309 to 325 and 534 to 551 was used (access number M61175). In order to synthesize the c-fos probe, a sense primer (5′ gac ttt tgc gca gat ctg tc 3′) and an antisense primer (5′ ctg ctc tac ttt gcc cct tc 3′) corresponding to respective nucleotide positions 276 to 295 and 508 to 527 of the cDNA was used (access number X06769).

In the PCR reaction, the genomic DNA was first denatured at 94° C. for four minutes, followed by 30 cycles of one minute at 94° C., one minute at 55° C. and one minute at 72° C. The extension reaction was carried out at 70° C. for 10 minutes. The amplified fragments were fractionated in a 1% agarose gel in 1×TAE buffer. Fragments corresponding to the expected lengths of the gene-specific probes were extracted using the QIAquick gel extraction kit from Qiagen. The expected lengths of the amplified fragments were 351 nucleotides (exon-III), 347 nucleotides (exon-IV), 243 nucleotides (exon-V) and 252 nucleotides (c-fos). These fragments were cloned into a PCR II Topo vector (Invitrogen), and their nucleotide sequences were verified using an automatic sequencer.

The plasmids were isolated using a QIAprep spin miniprep kit from Qiagen. In order to synthesize linearized plasmids for synthesis of sense riboprobes, the plasmids were first linearized with suitable restriction enzymes. Riboprobes were synthesized using Sp6, T3 or T7 RNA polymerases and labeled using rNTP mix which contains digoxigenin-labeled uridine triphosphates. All restriction enzymes, RNA polymerases and digoxigenin-labeled rNTPs were purchased from Roche Diagnostics. The in situ hybridization was carried out as described previously (Wiechers, B. et al. (1999), A changing pattern of brain-derived neurotrophic factor expression correlates with the rearrangement of fibers during cochlea development of rats and mice, J. Neurosci. 19, pages 3033 to 3042). The sections were covered with Moviol (Sigma) and examined using an Olympus AX70 microscope.

### Reverse Transcription, Semiquantitative PCR

Total RNA was isolated using the total RNeasy kit from Qiagen both from the cochlea and from brain tissue and was treated with DNase (Ambion) in order to remove DNA contaminants. The RNA was then purified and the concentration was determined by spectrometric measurements. Reverse transcription of the brain tissue was carried out in a reaction volume of 20 μl using 1 μg of total RNA as template and SUPERSCRIPT II Rnase H reverse transcriptase according to the Invitrogen protocol. Because of the limited amount of RNA in the cochlear tissues, Qiagen Senscript reverse transcriptase was used, as described in the Qiagen protocol, using 50 ng of total RNA as template. The number of cycles and the annealing temperature for the PCR was optimized so that the signals obtained for BDNF, c-fos and glyceraldehyde-3-phosphate dehydrogenase (GAPDH) were not saturated.

The PCR primer sequences for arc were 5′ caa tgt gat cct gca gat tg 3′ and 5′ tgt tgg cat agg ggc taa ca 3′; for BDNF they were 5′ ttc gac ccc gcc cgc cgt gg3′ and 5′ ccc ctt tta atg gtc agt gt 3; for c-fos they were 5′ gac ttt tgc gca gat ctg tc 3′ and 5′ att cct ttc cct tcg gat tc 3′; for GAPDH they were 5′ tct act gc gtc ttc ac acc a 3′ and 5′ agg aga caa cct ggt cct cag t 3′. The PCR was carried out in a final reaction volume of 25 μl, using both primers for GAPDH and the activity-dependent genes simultaneously. In this duplex PCR reaction, a housekeeping gene in the same PCR reaction represents an internal control, so that the intensity of the activity-dependent gene can be compared unambiguously with the control and the treated samples. PCR ready-to-go beads from Amersham Pharmacia were used in order to ensure a standardization of the PCR conditions and to reduce contamination during pipetting. Finally, the PCR products were analyzed in a 2% agarose gel which was visualized using an ethidium bromide stain and densitometric analysis using an Alpha Imager 2200 from Biometra. The intensity of the amplified activity-dependent gene was normalized for each reaction to the level of the coamplified GAPDH. The amplification conditions for arc, BDNF, c-fos and GAPDH were for the initial denaturation phase of 94° C. for three minutes, 30 cycles each of one minute of denaturation (94° C.), one minute of annealing (54° C.), 1.5 minutes of extension (72° C.) and a final extension phase of ten minutes at 72° C. The PCR fragments were cloned and sequenced as described previously.

### ABR Screening (ABR=Auditory Brainstem Response)

The anesthesia of the animals and the ABR screening was carried out as described previously; cf. Knipper, M. et al. (2000), loc. cit.; Schimmang, T. et al. (2003), Lack of BDNF and trkB signalling in the postnatal cochlea leads to a spatial reshaping of innervation along the tonotopic axis and hearing loss, Development 130, pages 4741 to 4750.

### Results

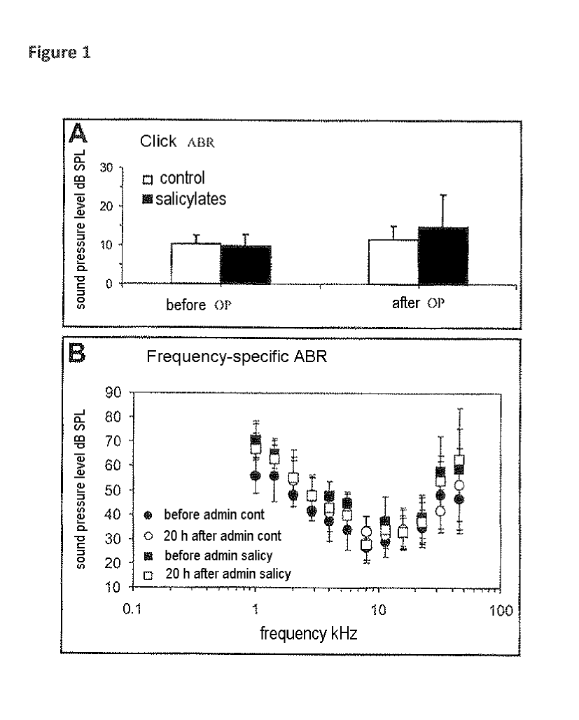

As described above, salicylate was administered locally in the niche of the round window to female rats of approximately the same weight. In order to preclude any post-traumatic effects on the expression of the activity-dependent genes in the cochlear neurons, the individuals used were exclusively those showing no change in the hearing capacity after application of gel foam. As shown in FIG. 1, no significant changes in the click-dependent ABR screening were found before or 20 hours after the operation.

The thresholds in the ABR screening were substantially identical before and after the operation (OP), and no substantial differences emerged either on administration of salicylate (n=13; 5 μl; 70 mg/kg; salicylate) or of artificial perilymph (n=9; 5 μl; control); cf. FIG. 1 (A). A frequency-specific ABR screening before and 20 hours after local administration of salicylate (n=5; 5 μl; 70 mg/kg; salicylate) or artificial perilymph (n=6; 5 μl; cont) into the niche of the round window shows that there is no loss of hearing; cf. FIG. 1(B).

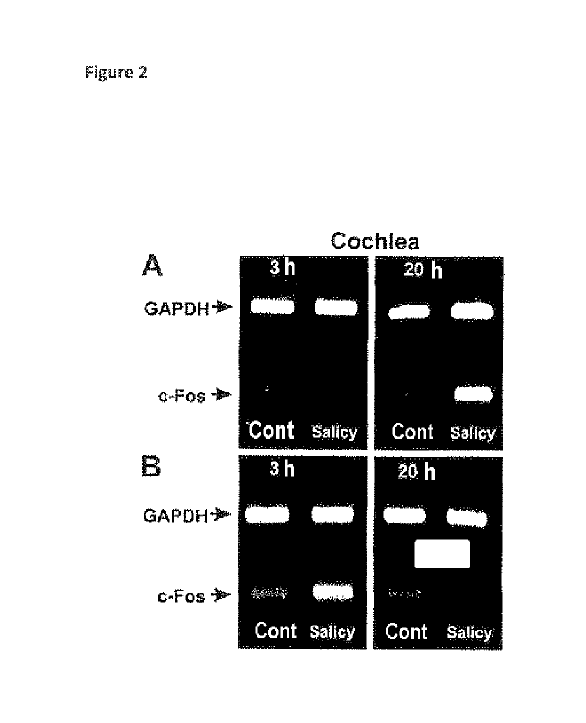

In a first approach to finding the suitable time at which either locally or systemically administered salicylate reaches the cochlea and modifies the neuronal excitability, c-fos expression was investigated in cochlear tissue by RT-PCR analysis at various times after the salicylate administration. It was found that gel foam administration of salicylate (5 μl, 70 mg/ml) did not influence c-fos expression before about 20 hours, possibly because of a slow secretion of the liquid from the gel foam. A significant upregulation of c-fos was found 20 hours, but not three hours, after administration (FIG. 2A). In contrast thereto, upregulation of c-fos was observed after only three hours, without significant changes at longer times of up to 20 hours, by systemic administration of salicylate (350 mg/kg of body weight) by intraperitoneal injection. GAPDH expression is identical both in the control approach and in the salicylate-treated approach, demonstrating that equal amounts of RNA (50 ng) were used in these experiments (FIG. 2B). In order to compare corresponding acute effects, subsequently the effects of locally administered salicylate were investigated 20 hours, and of systemically injected salicylate 3 hours, after administration.

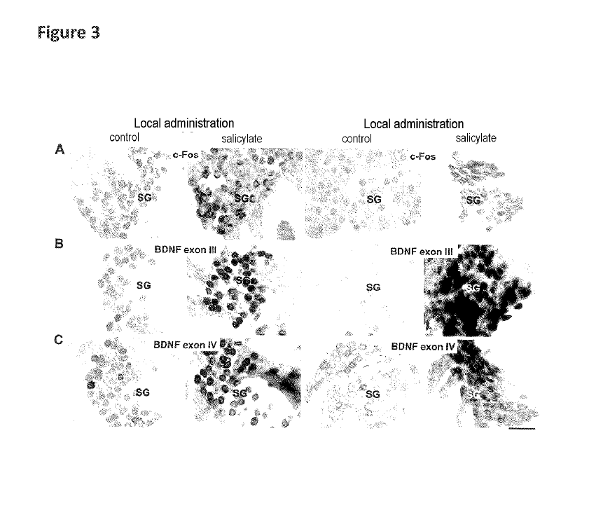

In a further step, the effect of locally and systemically administered salicylate on the expression of certain BNDF splice variants in the cochlear spiral ganglia (SG) of the rat were investigated by in situ hybridization. Expression of c-fos was observed for comparison. As shown by way of example in FIG. 3 for three individual experiments in duplicates, the hybridization signals for c-fos (FIG. 3A), BNDF exon-III (FIG. 3B) and BDNF exon IV (FIG. 3C) in the middle basal cochlear turn were significantly increased (dark color) compared with administration of the same volume of artificial perilymph (FIG. 3, control) after both local (left, 5 μl; 70 mg/ml; local administration) and systemic (right, 350 mg/kg of body weight; systemic administration) administration of salicylate. No hybridization signals were observed on use of sense samples (data not shown). BDNF expression showed a characteristic decrease along the tonotopic axis from the basal/middle basal to the more apical cochlear turns (Schimmang. T. et al. (2003) loc. cit.) and the salicylate-induced increase in the expression pattern influenced primarily the more basally located cochlear turns.

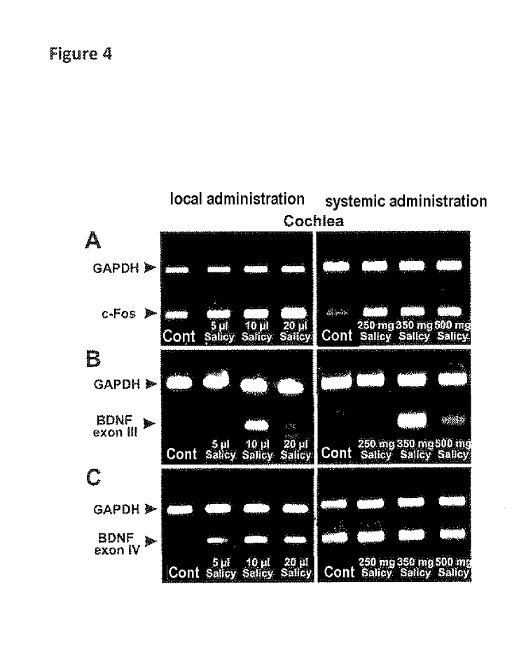

In order to investigate possible differences in the activation patterns of certain BDNF transcripts at various levels of excitation, the effect of various salicylate dosages which might influence neuronal excitability differently was analyzed; cf. Kumagai (1992), Effect of intravenous injection of aspirin on the cochlea, Hokkaido Igaku Tasshi 67 (2), pages 216 to 233; Stypulkowski (1990), Mechanisms of salicylate ototoxicity, Hear Res. 46 (1-2), pages 113 to 145. For this purpose, the effect of locally administered gel foam impregnated with various volumes (5, 10, 20 μl) of 70 mg/ml salicylate and, in parallel, the effect of various concentrations of systemically administered salicylate (250 mg/kg, 350 mg/kg, 500 mg/kg of body weight) was investigated. Semiquantitative RT-PCR analysis of the total RNAs was used to investigate the c-fos, BDNF exon-III and BDNF exon IV transcripts in cochlear tissue (FIG. 4).

It is of interest that a dose-dependent effect of salicylate was found with both methods of administration and led to slight changes, compared with GADPH, in the upregulation of c-fos expression. The greatest effects were found on administration of 5 μl or 10 μl of salicylate on the round window or on injection of 250 mg/kg or 350 mg/kg salicylate, whereas less distinct effects were found with higher concentrations in both methods of administration (FIG. 4A).

Use of the same template to amplify either BDNF exon-III or -IV revealed a distinct difference in the activation pattern of the various activity-dependent genes.

Irrespective of the method of administration, BDNF exon-III was activated with some delay after BDNF exon-IV, resulting in a peak of BDNF exon-III expression with higher salicylate concentrations (FIG. 4B), whereas both c-fos (FIG. 4A) and BDNF exon-IV (FIG. 4C) respond to lower doses of salicylate. Comparable results were obtained in three experiments in duplicates which confirmed the densitometric analysis which is shown in FIG. 4B, according to which there is a significant increase in the expression of BDNF exon-IV (49±12%, n=8, p<0.05) and of c-fos (69±11%, n=8, p<0.05).

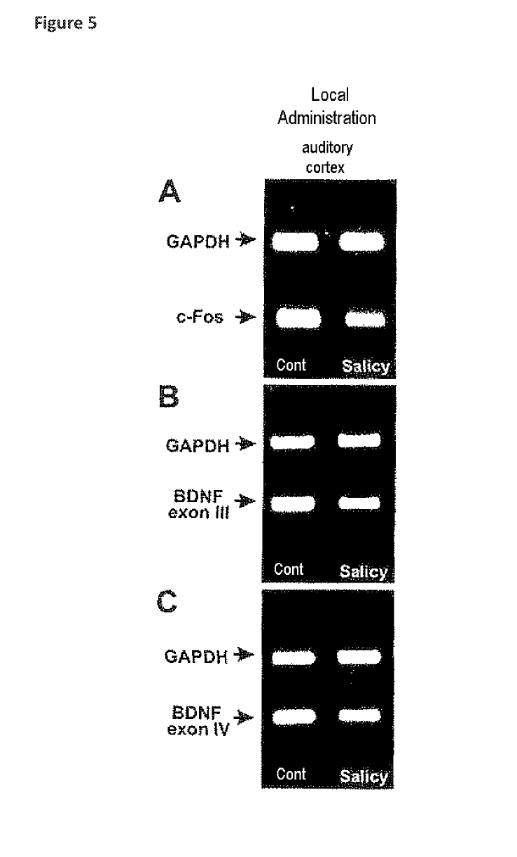

In the auditory cortex, the BDNF exon-III and -IV splice variants, the common BDNF exon-V and c-fos transcripts were amplified (data not shown). The auditory cortices investigated were obtained from animals in which the cochlea were investigated for the dose-dependent effect of salicylate, the mRNA was isolated and an RT-PCR was carried out as described under Material and Methods.

Whereas locally or systemically administered salicylate leads to an increase in the expression of the investigated activity-dependent genes in the cochlea (FIG. 4), it was found in three independent experiments in duplicates that the two methods lead to opposite effects on the activity-dependent genes in the auditory cortex. In contrast to the effect on the cochlear neurons, local administration of all dosages of salicylate led to a reduction in the expression of c-fos (FIG. 5A, left), of BDNF exon-III (FIG. 5B, left) and BDNF exon-IV (FIG. 5C, left).

Whereas a different activation pattern was found in the cochlea for different dosages and different BDNF transcripts, the decreasing effect at higher concentrations was not so evident in the auditory cortex (FIG. 5A-C). It was confirmed by densitometric analysis that there is a significant decrease in the expression of BDNF exon-IV (49±12%, n=8, p<0.05) and c-fos (69±11%, n=8, p<0.05).

In a further experiment, the inventors investigated whether the phenomenon of elevated BDNF expression, which was found for the first time in acute tinnitus, can be abolished by using isradipine, an L-type Ca⁺⁺ channel antagonist, and thus a corresponding substance is suitable for the treatment of acute tinnitus, preferably on local administration, or of phantom pain.

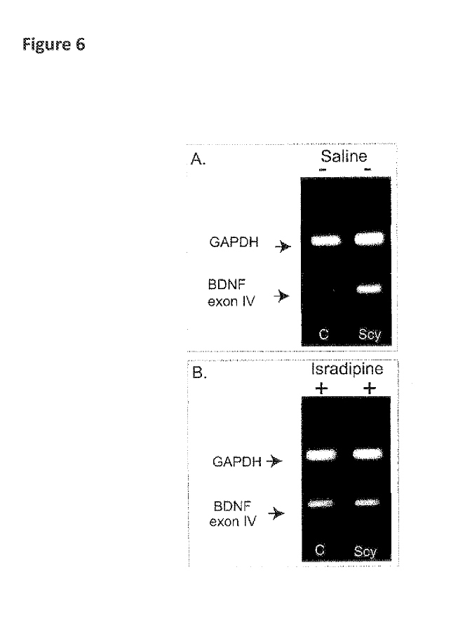

For this purpose, 22 hours before the removal of tissue, 10 μl of 0.9% strength saline solution (FIG. 6A, saline) and 10 μl of a 10 mM isradipine solution (FIG. 6B, isradipine) were administered locally into the niche of the round window, i.e. in front of the round window membrane. 3 hours before the removal of tissue on the cochlear, an identical volume of saline solution (C) or 350 mg/kg of body weight salicylate (Scy) was injected systemically. After the removal of tissue, the expression of BDNF was analyzed in both approaches.

FIG. 6 depicts the expression of BDNF exon IV under the aforementioned conditions. As expected, salicylate brings about upregulation of BDNF in the cochlear neurons (FIG. 6A, right-hand lane), whereas upregulation of BDNF is inhibited in the identical approach in the animal group in which isradipine was administered instead of saline.

The experiment described above and depicted in FIG. 6 was carried out under identical conditions also for a further L-type Ca⁺⁺ channel antagonist, nifedipine. In this case, there was likewise detected to be an inhibitory effect on BDNF expression, which was elevated after the induction of acute tinnitus, in the cochlear neurons, although less strongly than with isradipine (data not shown).

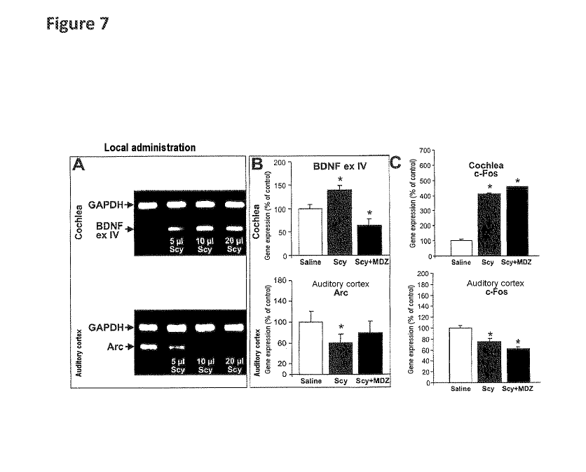

The inventors have also investigated whether the elevated BDNF expression which is associated with acute tinnitus can also be inhibited by administering GABA receptor agonists such as benzodiazepines. As already described above, increasing amounts of salicylate (Scy) was administered locally into the niche of the round window of female rats. It emerged in this case, as expected on the basis of the experiments discussed above, that expression of the BDNF exon-IV transcript was increased in the cochlear neurons, and expression of the activity-dependent cytoskeletal protein Arc was decreased in the auditory cortex. The result of a representative RT-PCR is shown in FIG. 7A for n=3 with comparable result.

In a further experimental approach, 350 mg/kg salicylate was administered systemically to the rats. As shown in FIG. 7A for local round window administration, systemic administration of salicylate also leads to an elevated expression of BDNF exon-IV (FIG. 7B, top) and, as expected, of c-fos (FIG. 7C, top) in cochlear neurons, whereas a decrease in the expression of Arc (FIG. 7B, bottom) and BDNF exon-IV (not shown) and occasionally of c-fos (FIG. 7C, bottom) is detectable in the primary auditory cortex.

Two and half hours after the induction of acute tinnitus by injection of 350 mg/kg salicylate, the animals received systemic administration of midazolam (Dormicum, Roche, Grenzach-Wyhlen, Germany) (0.5 mg/kg of body weight), and gene expression was investigated after removal of organs with the aid of RT-PCR. It emerged from this that midazolam (MDZ) leads to a significant reduction in the effect of salicylate on the expression of BDNF exon-IV (n=7) in the cochlear neurons and of Arc in the auditory cortex (FIG. 7B, right-hand bar, n=12), but the expression of c-fos remains incapable of being influenced (FIG. 7C, right-hand bar, n=7 to 12). Statistical analysis was carried out with Student's T test; *=p<0.05.

In a further experiment corresponding to the above, a GABA receptor agonist was administered, under conditions which were otherwise identical, not systemically but locally into the niche of the round window. It emerged from this that the elevated BDNF exon-IV expression connected with acute tinnitus was inhibited even more greatly in the cochlear neurons, and the effects on the expression of BDNF exon-IV in the auditive cortex were distinctly reduced.

Local administration of a BDNF antagonist consequently abolishes the pathological dysregulation of BDNF expression which is observed in tinnitus, such as acute, subacute and chronic tinnitus, and phantom pain. The inventors were thus able to demonstrate that BDNF antagonists are substances suitable in principle for the treatment of tinnitus, such as acute, subacute and chronic tinnitus.

In a further experiment, a trkB antagonist (hereafter designated as “AN”), i.e., a cyclic peptide binding to a functional site on trkB, which was identified from the NT-4/trkB crystal structure (Banfield et al., 2001, Structure 9(12): 1191-1199), has been tested for its ability to attenuate or suppress tinnitus. For this purpose, a behavioural model as described by Rüttiger et al. (2003, Hearing Research 180(1-2): 39-50) was used to discriminate between animals with and without tinnitus.

27 adult female Wistar rats were trained in a conditioning chamber to actively access a liquid feeder whenever a constant sound was present. During silence, no reward was given. The conditioning was completed when animals performed more accesses to the reward feeder during periods of sound than during periods of silence. Frequencies of feeder access action of a rat were calculated for periods of sound and periods of silence separately (number of accesses/min) and normalized.

Silence activity ratio (SA ratio)=n_{silence}/t_{silence}(n_{sound}/t_{sound})⁻¹; SA ratio≧0; wherein n is the number of accesses to a reward feeder during silence and sound (n_{silence} and n_{sound}, respectively) and t is the time of silence and sound being played (t_{silence} and t_{sound}, respectively) for a given animal. Based on previously generated experimental data, an SA ratio (“tinnitus score”) of 0.2 or higher seems to be a reliable indicator of tinnitus perception.

On Day 0, the baseline SA ratio for conditioned rats was determined. Animals were then anesthetized; 13 of them were exposed intra-aurally for 2 hours to a continuous 10 kHz tone at an intensity of 120 dB SPL in a sound attenuation booth. This exposure is susceptible of inducing tinnitus in rats (e.g. Tan et al., 2007, Journal of Neuroscience 145: 715-726). The acoustic stimulus was calibrated at the head level of the animal. The remaining 4 animals were placed in the sound attenuation booth for the same duration, but the speaker remained turned off (sham exposure).

All noise exposed animals were treated bilaterally 1 hour after noise trauma under anesthesia with either artificial perilymph (AP, n=5) or AN at a concentration of 250 μM (n=17). First, auditory brainstem measurements were performed. For the local treatment administration, the fur was removed behind the ears and the bulla exposed in a retro-auricular approach. A small hole was carefully drilled into the bony bulla just above the round window niche (0.6-1 mm in diameter). The mucosa was opened and the region around the round window carefully dried of fluid. Through the hole, a small gelfoam pellet was deposited into the round window niche. 5-8 μl of AP or AN in AP solution was applied on the gelfoam by means of a precision pipette with gel loader tips, thus avoiding air bubbles to remain under the gel. Visual inspection showed that the niche was completely filled and covered with the gel. The hole in the bulla was then covered from the outside with muscular tissue and the wound was sutured with surgical thread (Vicryl, Johnson & Johnson, Norderstedt, Germany). Postoperatively the animals were kept warm with the body temperature being controlled until they woke up.

The SA ratio was determined on Day 14 (14 days after the real or mock sound exposure) again, and the change from baseline calculated. All animals were sacrificed thereafter.

The mean SA ratio (tinnitus score) of AP-treated control animals exposed to 120 dB SPL at 10 kHz for 2 hours increased 14 days post exposure from 0.02 to 0.88. The increase was statistically significant compared to sham exposed animals according to the Mann-Whitney-U-test (p<0.01), demonstrating the presence of tinnitus. Administration of the trkB antagonist AN attenuated the increase in the tinnitus score compared with AP treated animals in a significant fashion (p<0.01). This outcome demonstrates that inhibition of the BDNF signal transduction cascade through blocking of trkB allows for treating tinnitus.

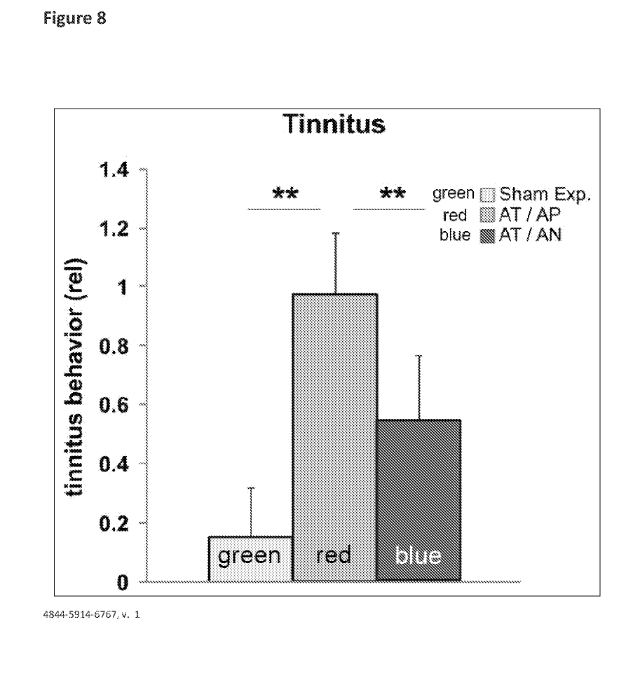

FIG. 8 shows the change in the silence activity ratio (SA ratio, tinnitus behavior) in conditioned animals from baseline prior to exposure to noise (120 dB SPL at 10 kHz for 2 hours) or sham exposure to 14 days post exposure. An increase of the SA ratio due to animals performing more frequently an activity in silence which they were conditioned to perform only upon an acoustic stimulus means that they perceive a similar sound internally, i.e. tinnitus. Animals treated with artificial perilymph only (n=5) experienced a large increase in tinnitus behavior that was significantly higher than in sham exposed control animals (Mann-Whitney-U-test; p<0.01). In contrast animals treated with the trkB antagonist AN showed an increase in tinnitus behavior that was statistically significantly lower than the AP treated animals (p<0.01). This outcome shows that inhibition of the BDNF signal transduction cascade is effective in attenuating or suppressing tinnitus.

The inventors were able to show for the first time that effective treatment of tinnitus, such as acute, subacute and/or chronic tinnitus, and phantom pain is possible with substances which interact with the BDNF signal transduction cascade or inhibit it.

1. A method for the treatment of the phantom phenomena of tinnitus and/or of phantom pain in a human or animal being, comprising administering to the human or animal being an effective amount of a trkB antagonist thereby treating the phantom phenomena of tinnitus or of phantom pain in the human or animal being.

2. (canceled)

3. (canceled)

4. The method of claim 1, wherein the trkB antagonist is administered locally on or in the ear or at the amputation site.

5. (canceled)

6. (canceled)

7. The method of claim 4, wherein local administration of the trkB antagonist in the case of treating tinnitus is performed by using a micrometering system.

8. The method of claim 4, wherein local administration of the trkB antagonist is performed by use of a biodegradable hydrogel, which serves as carrier matrix for the trkB antagonist.

9. The method of claim 4, wherein the trkB antagonist is administered locally to the cochlear ganglia.

10. (canceled)

11. The method of claim 1, wherein the trkB antagonist binds directly to the BDNF receptor (trkB).

12. The method of claim 1, wherein the trkB antagonist is administered locally using an implantable drug delivery system for treating tinnitus in a patient.

13. (canceled)

14. The method of claim 12, wherein trkB antagonist is administered locally onto the round window of the inner ear.

15-17. (canceled)

18. The method of claim 1, wherein the method further comprises determining the level of BDNF expression in the auditory system of the patient.

19. The method of claim 1, wherein the method further comprises determining the level of BDNF expression in the auditory cortex of the patient.

20. The method of claim 1, wherein the method further comprises determining the level of BDNF expression in the cochlear ganglia of the patient.

21. (canceled)

22. (canceled)

23. A method of inhibiting BDNF signal transduction or blocking the BDNF receptor (trkB) in the cochlear ganglia of a patient suspected of having tinnitus comprising administering to the patient an effective amount of a composition comprising a trkB antagonist, wherein the trkB antagonist is administered locally, and wherein inhibiting BDNF signal transduction or blocking the BDNF receptor (trkB) results in an amelioration of tinnitus symptoms.

24. The method of claim 23, wherein the trkB antagonist inhibits the trkB ligand from binding to the receptor.

25. The method of claim 23, wherein the trkB antagonist directly binds to trkB.

26-40. (canceled)

41. A method for the treatment of the phantom phenomena of acute, subacute and/or chronic tinnitus in a human or animal being, comprising administering to the human or animal being an effective amount of a trkB antagonist thereby treating the phantom phenomena of tinnitus in the human or animal being.

42. (canceled)

43. The method of claim 41, wherein the phantom phenomena is subacute or chronic tinnitus.

44. The method of claim 41, wherein the trkB antagonist inhibits the binding of the trkB ligand to trkB.

45. The method of claim 41, wherein the trkB antagonist is administered locally to the cochlear ganglia.

46. The method of claim 41, wherein trkB antagonist is administered locally onto the round window of the inner ear.


SEQUENCE LISTING (16 sequences)

SEQ ID NO: 1 - DNA, 20 bp, Rattus sp.
acccactttcccattcaccg

SEQ ID NO: 2 - DNA, 20 bp, Rattus sp.
cctttttcagtcactacttg

SEQ ID NO: 3 - DNA, 20 bp, Rattus sp.
ccaatcgaagctcaaccgaa

SEQ ID NO: 4 - DNA, 20 bp, Rattus sp.
tcagggtccacacaaagctc

SEQ ID NO: 5 - DNA, 17 bp, Rattus sp.
gaggaccagaaggttcg

SEQ ID NO: 6 - DNA, 18 bp, Rattus sp.
tttatctgccgctgtgac

SEQ ID NO: 7 - DNA, 20 bp, Rattus sp.
gacttttgcgcagatctgtc

SEQ ID NO: 8 - DNA, 20 bp, Rattus sp.
ctgctctactttgccccttc

SEQ ID NO: 9 - DNA, 20 bp, Rattus sp.
caatgtgatcctgcagattg

SEQ ID NO: 10 - DNA, 20 bp, Rattus sp.
tgttggcataggggctaaca

SEQ ID NO: 11 - DNA, 20 bp, Rattus sp.
ttcgaccccgcccgccgtgg

SEQ ID NO: 12 - DNA, 20 bp, Rattus sp.
ccccttttaatggtcagtgt

SEQ ID NO: 13 - DNA, 20 bp, Rattus sp.
gacttttgcgcagatctgtc

SEQ ID NO: 14 - DNA, 20 bp, Rattus sp.
attcctttcccttcggattc

SEQ ID NO: 15 - DNA, 20 bp, Rattus sp.
tctactgcgtcttcacacca

SEQ ID NO: 16 - DNA, 22 bp, Rattus sp.
aggagacaacctggtcctcagt